(19) United States
(12) Patent Application Publication
(10) Pub. No.: US 2018/0235256 A1
(43) Pub. Date: Aug. 23, 2018

(54) PROCESS FOR PRODUCING PROTEIN CONCENTRATE AND A CELLULOSIC RESIDUE MATERIAL FROM RICE BRAN

(71) Applicant: Zea10,LLC, Eden Praire, MN (US)

(72) Inventors: Ian Mackay, Eden Praire, MN (US); Karl Greden, Hinckley, MN (US)

(21) Appl. No.: 15/952,142

(22) Filed: Apr. 12, 2018

Related U.S. Application Data
(63) Continuation-in-part of application No. 15/171,780, filed on Jun. 2, 2016.
Application No. 15/171,780 is a continuation-in-part of application No. 14/591,904, filed on Jan. 7, 2015, now abandoned.

(51) Int. Cl. A23K 10/14; A23J 1/12; A23J 3/34; A23K 10/38; A23K 50/75; A23K 50/10; C12P 21/06; C12P 19/00; C12P 19/14; C10L 1/02

(52) U.S. Cl. CPC A23K 10/14; A23J 1/125; A23J 3/348; A23K 10/38; A23K 50/75; A23K 50/10; C10L 2200/0469; C12P 19/00; C12P 19/14; C12Y 302/01001; C12Y 304/21062; C10L 1/02; C12P 21/06

(57) ABSTRACT

A process for treating rice bran, preferably defatted rice bran, to produce a high value protein product and a cellulosic residue both from rice bran. The high value protein product is useful as a protein supplement or feed for livestock and poultry and the cellulosic residue has value as a feedstock for a thermochemical process unit for the production of a biofuel. The rice bran is subjected protein hydrolysis and a resulting liquid stream containing hydrolyzed proteins is sent through a two membrane filtration stages, the first being a microfiltration and the second being a nanofiltration stage.

### CROSS-REFERENCE TO RELATED APPLICATIONS

This application is a Continuation-In-Part of application Ser. No. 15/171,780 filed Jun. 2, 2016, which is a Continuation-In-Part of Ser. No. 14/591,904 filed Jan. 7, 2015 which is based on Provisional Application 61/924,678 filed on Jan. 7, 2014.

### FIELD OF THE INVENTION

This invention relates to a process for treating rice bran, preferably defatted rice bran to produce a high value protein product and a cellulosic residue material. The high value protein product is useful as a food protein supplement or as feed for livestock and poultry. The cellulosic residue material has value as a feedstock for a thermochemical process unit for the production of biofuel.

### BACKGROUND OF THE INVENTION

Rice bran is a by-product of the rice milling process (the conversion of brown rice to white rice) and contains various antioxidants that impart beneficial effects on human health. Defatted rice bran is what remains after the bran has been removed from the whole rice grain and has had most of the oil removed. It contains protein (up to 18 wt. %), dietary fiber (up to 32 wt. %), crude fiber (up to 13 wt. %) ash (up to 18 wt. %), residual fat (up to 5 wt. %), and moisture (up to 14 wt. %). DRB is found throughout the world since rice is a staple grain and the oil obtained from the bran is valuable, but defatted rice bran itself has limited market value as is. There is a need in the art for more economical and efficient processes for obtaining maximum value from waste biomass, such as rice bran. Over 600 million tons of rice is harvested on a global scale. Much of the nutritional value of rice lies in the bran and germ, which is comprised primarily of the bran layer and germ of the rice with some fragments of hull and broken rice. Rice bran is typically comprised of protein, fat, carbohydrates and contains micro-nutrients such as vitamins, minerals, anti-oxidants and phytosterols. The high oil content of rice bran makes it subject to rancidification and is typically discarded during the milling process or used as low-value animal feed.

### SUMMARY OF THE INVENTION

In accordance with the present invention there is provided a process for producing a protein product suitable for human consumption and a cellulosic product suitable as a feedstock for thermochemical processing from rice bran containing a starch component and a protein component, which process comprises:

a) introducing into a hydrolysis reactor, with constant stirring, rice bran an effective amount of water, so that a slurry is formed having a ratio of water to rice bran, by weight, of from about 8:1 to about 11:1;

b) heating said slurry to a temperature from about 30° C. to about 70° C.;

c) providing that the pH of the slurry be in the range from about 7 to about 10.5;

d) introducing into said hydrolysis reactor an effective amount of alkaline protease enzyme;

e) maintaining hydrolyzing condition, including temperatures from about 30° C. to about 70° C. and a pH of about 7 to 10.5, for an effective amount of time to allow the degree of hydrolysis of proteins to reach between 1 to 10;

f) raising the temperature an effective amount to result in deactivating the protease enzyme;

g) conducting the slurry resulting from step f) above to a liquid/solids separation stage resulting in a wet-solids fraction comprised of cellulosic residue material, and a liquid fraction comprised of water, protein, oils, sugars, fiber and ash;

h) conducting said aqueous fraction to a membrane microfiltration stage containing a membrane capable of performing a separation in the 20 to 500 kDa size range thereby resulting in a retentate comprised of cellulosic residue material and a permeate comprised of water and hydrolyzed protein products;

i) conducting said permeate to a membrane nanofiltration stage containing a membrane capable of performing a separation in the 500 to 1500 Daltons size range, thereby resulting in a retentate comprised of hydrolyzed protein products, and a permeate comprised of an aqueous solution of water-soluble constituents, including monosaccharides, resulting from the above process steps;

j) spray drying said retentate resulting in a spray dried hydrolyzed protein product; and

k) drying said wet-solids fraction resulting from step g) above to produce a dried cellulosic residue product.

In a preferred embodiment, the rice bran is defatted rice bran.

In another preferred embodiment, after heating step b) but before the alkaline protease introduction step d), the pH is adjusted to 4.5 to 8 and an effective amount of a starch hydrolyzing enzyme is added to the slurry, wherein at least a fraction of the starch is hydrolyzed to monosaccharides at starch hydrolysis conditions including temperatures from about 30° C. to 70° C. After starch hydrolysis the pH is adjusted to 7 to 10.5 and step d) is continued by adding an effective amount of an alkaline protease enzyme.

In another preferred embodiment, the rice bran, after being treated with water, is subjected to an effective amount of ultrasonic energy capable of improving the accessibility of proteins of the rice bran.

### BRIEF DESCRIPTION OF THE FIGURES

FIG. 1 hereof is a simplified flow scheme of one preferred embodiment of the process of the present invention for producing a protein rich product and a cellulosic residue material from rice bran. This figure shows an optional stage (milling) for reducing the average particle size of the rice bran in the event it is received having an average particle size too large for the instant process.

FIG. 2 hereof is a simplified flow scheme of another preferred embodiment of the present invention showing both an optional milling stage and an optional sonication stage. Also, a base solution can be used to hydrolyze proteins instead of a protease enzyme.

### DETAILED DESCRIPTION OF THE INVENTION

As previously mentioned, rice bran is a nutrient-dense by-product from the milling of rice. Unprocessed rice bran will typically be comprised of about 18 to 23 wt. % carbohydrates other than starch, about 18 to 30 wt. % starch, about 15 to 18 wt. % proteins, and about 18 to 23 wt. % fats (oils). Rice bran suitable for the practice of the present invention, is rice bran wherein as much of the fat is removed as possible by any suitable method. The term “fat” or “fats” as used herein with respect to rice bran also includes the term “oil” or “oils”. A particular suitable method for removing fats from rice bran is solvent extraction, which can include supercritical solvent extraction. Solvent extraction is well known in the art. Preferred solvents include the C3 to C6 alkanes, preferably propane and hexane. The term “rice bran” as used herein means a rice bran that has gone through a defatting process, such as solvent extraction, and contains no more than about 3 wt. % fat, preferably from about 0.1 to 3 wt. %, preferably from about 0.2 to about 3 wt. %, more preferably from about 0.5 to about 2.5 wt. %, and most preferably from about 0.5 to about 2.0 wt. % fat. These weight percents are based on the total weight of the rice bran excluding water.

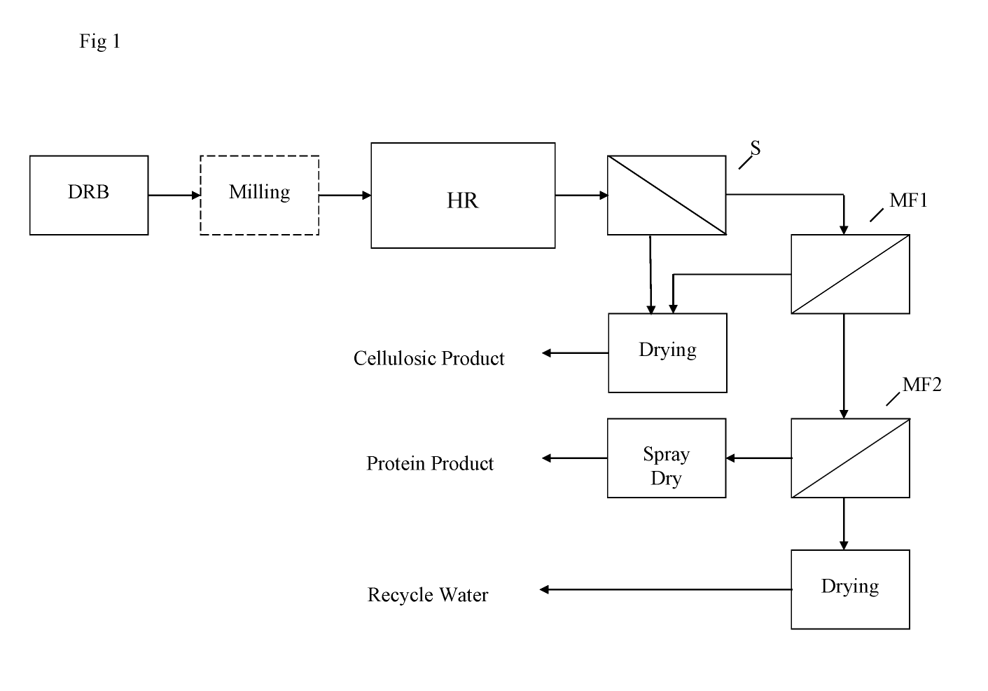

The present invention can be better understood with reference to the figures hereof. FIG. 1 hereof is one preferred embodiment wherein dry rice bran DRB can be milled to reduce its average particle size if necessary. It is preferred, for purposes of the instant process, that the rice bran have an average particle size of less than about 500 microns. The rice bran is then introduced into hydrolyzing reactor HR along with an effective amount of water. By “effective amount of water” we mean at least that amount of water needed to make a slurry that can be efficiently mixed in a conventional stirred tank reactor so that insoluble and soluble components stay in contact during hydrolysis. Such an effective amount of water will be from 8 to 1 to 11 to 1, preferably from about 9 to 1 to 10 to 1 water to rice bran, on a dry weight basis.

The slurry is heated to a range of about 30° C. to about 70° C. and the pH adjusted to be in the range of about 7 to 10.5. An effective amount of alkaline protease enzyme is added. By “effective amount” we mean at least that amount of alkaline protease enzyme that will be capable of solubilizing at least 80 wt. % of the proteins, preferably from about 80 wt. % to 90 wt. % of the proteins. This amount will typically be from about 0.1 wt. % to about 0.3 wt. %, preferably about 0.25 wt. %, on a dry weight basis. The protease enzyme is added while maintaining the above temperature and pH ranges and hydrolysis is allowed to take place until the degree of protein hydrolysis is between about 1 and 10, preferably between about 2 and 8, and more preferably between about 3 and 4.5.

At this point, the temperature is raised to an effective temperature capable of deactivating the alkaline protease enzyme, but not so high as to result in an undesirable effect. This temperature will typically be from about 75° C. to 100° C., preferably from about 85° C. to about 90° C. The mixture is then held at that temperature for an effective period of time to ensure that the enzyme is deactivated. This effective amount of time will typically be from about 2.5 to about 30 minutes, preferably from about 5 to 20 minutes, more preferably about 15 minutes.

The resulting protease enzyme treated rice bran slurry is then conducted from reactor HR to a liquid/solids separation stage S resulting in a liquid fraction comprised of water, hydrolyzed proteins, minor amounts of other water-soluble constituents, and hydrolyzed starch in the event that the optional starch hydrolysis step was used prior to protein hydrolysis. There will also be a wet-solids fraction comprised of the remaining rice bran material, preferably a cellulosic residue material having a substantially reduced level of proteins. The term “wet-solids” as used herein means a non-fluid solids slurry comprised of about 50 wt. % to about 85 wt. % water. Non-limiting examples of other water-soluble constituents include ash, salts, sugars, starches, and dietary fibers. It is preferred that the separation stage include use of a centrifuge. The resulting separated wet-solids can become part of the solids stream which is sent to a drying stage, or it can be sent independently to a drying stage.

The liquid fraction resulting from separation stage S is further processed to isolate proteins from insoluble constituents by conducting it to first membrane filtration stage MF1 which preferably contains one or more membranes having pores in the microfiltration size range of about 20 to 500 kDa. It is preferred that the pore be about 400 kDa, more preferably about 500 kDa. The selection of the precise upper size limit will be dependent on the membrane that provides the optimal protein transmission and flux. The filtration will preferably be conducted using micrometer sized cylindrical through pores that pass through the membrane at a 90° angle. Membrane filtration is well known in the art, therefore no detailed discussion of it is necessary in this document. Diafiltration is preferably used so that most of the proteins are in the permeate. Diafiltration is well known in the art and typically uses ultrafiltration membranes to remove, or to at least lower, the concentration of salts or solvents from solutions that contain proteins, peptides, nucleic acids, and other biomolecules. The retentate from membrane filtration stage MF1 will contain up to about 20% solids at the end of the filtration stage. The retentate can also be comprised of fats, fibers, and possibly a small amount of high molecular weight proteins. The retentate can be added to the wet-solids from separation stage S for drying, or it can be sent independently to a drying stage.

The protein-rich permeate from this first membrane filtration stage MF1, which will also contain other water-soluble constituents, is conducted to second membrane filtration stage MF2 which contains a nanofiltration membrane to further purify and dewater the proteins. Second membrane filtration stage MF2 will have a molecular weight cutoff of about 250 to 2000 Daltons, preferably from about 500 to 1000 Daltons. Diafiltration is preferably used to demineralize the retentate of second membrane filtration stage MF2 as well as to remove sugars from the retentate. The permeate of second membrane filtration stage MF2 will contain inter alia, salts, ash, sugars, and relatively low molecular weight proteins, peptides and amino acids. The retentate will have a solids content of about 15 to 25 wt. %, preferably greater than about 20 wt. %, and will be comprised of a protein isolate. This resulting protein isolate solution is spray dried in spray drying stage SD resulting in a substantially dry protein product, comprised of at least about 80 wt. % protein, preferably at least about 85 wt. % protein.

The permeate from membrane filtration stage MF2 is preferably conducted to reverse osmosis stage RO wherein substantially all, that is at least about 95 wt. %, preferably at least about 98 wt. %, of water-soluble constituents are removed to produce a recycle water RW stream.

The solids fraction from both the separation stage and first membrane filtration stage MF1 are dried to result in a cellulosic residue product that is suitable as a feed source for both humans and livestock and as feedstock for a thermochemical process that can be converted into a transportation or other fuel.

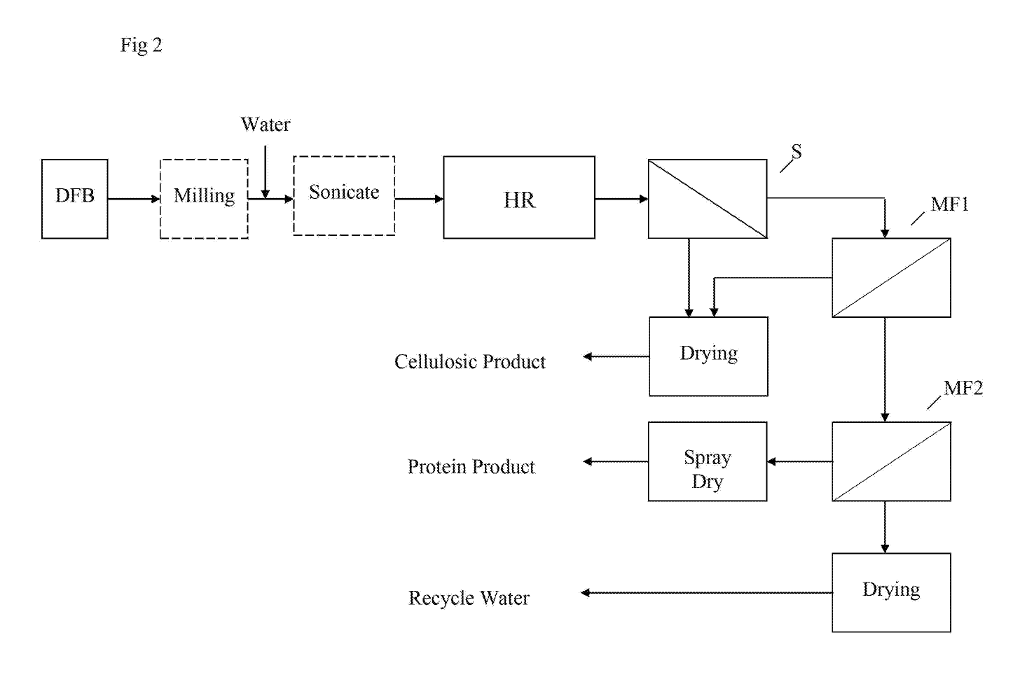

Reference is now made to FIG. 2 hereof which represents another preferred embodiment of the present invention for processing rice bran to produce a protein concentrate or isolate product and a protein-lean residue (cellulosic) that can be used as a feed component for humans or livestock, or as feedstock for a thermochemical process to produce a biofuel. By protein-lean we mean that the cellulosic residue will contain as low a level of proteins as possible. For example, the cellulosic residue will contain less than 2 wt. %, preferably less than 1 wt. % and more preferably less than about 0.5 wt. % based on the total weight of the cellulosic residue. In this embodiment, the rice bran is also optionally milled in the event it is received with too large an average particle size for the instant process. An effective amount of water is added to the rice bran, preferably at a ratio of 9:1 to 10:1 water to dry bran and the resulting rice bran slurry is optionally subjected to sonication to help with protein removal. We believe ultrasonic energy helps to break down cell structures, thereby improving access to proteins of the rice bran. It is preferred that the ultrasonic energy input be from about 3 to about 30 Joules/gram of rice bran with a frequency of about 40 kHz, with about 3 to about 10 Joules/gram being preferred. It will be understood that ultrasonic energy can also be used in the process represented in FIG. 1 hereof.

The protein-lean cellulosic residue is collected and can be marketed as a livestock feed component, or as a feedstock component for a subsequent thermochemical process, such as pyrolysis or gasification, that can be used for the production of biofuel, preferably a transportation fuel, more preferably a distillate fuel.

It is within the scope of this invention that a starch hydrolysis step be conducted prior to the protein hydrolysis step. In the case of starch hydrolysis, the slurry will preferably be adjusted to be in the pH range of about 4.5 to 8.0, more preferably from about 6.5 to 7.5 instead of a pH range of 7 to 10.5 that is preferred for protein hydrolysis. At least a portion of the starch of the rice bran is hydrolyzed by use of an effective amount of a starch hydrolyzing enzyme, preferably an amylase enzyme. By effective amount of starch hydrolyzing enzyme, we mean at least that amount needed to convert the starch, or apparent starch, content by about 80% to 99%, preferably from about 90% to 99% to monosaccharides. Any suitable amylase enzyme can be used in the practice of the present invention. Non-limiting examples of amylase enzymes that can be used in the practice of the present invention include fungal alpha-amylase, bacterial alpha-amylase, and fungal glucoamylase. Fungal glucoamylase enzymes are preferred. The amylase enzyme treated rice bran is subjected to hydrolysis conditions to cause the starch and apparent starch to hydrolyze to monosaccharides, thus resulting in molecules small enough to be membrane separated from the hydrolyzed protein moieties extracted in the following step. The amylase enzyme will preferably be used as an aqueous solution of an effective concentration of about 0.1 to 1 wt. %, preferably from about 0.2 to 0.4 wt. %, based on the dry weight of the rice bran.

After starch hydrolysis the pH of the slurry will be adjusted to 7 to 10.5 as in step c) above, and an effective amount of alkaline protease enzyme is added as in step d0 above, and hydrolyzing conditions maintained until the degree of hydrolysis of proteins is reached between 1 and 10. Steps e) through l) are resumed to obtained the desired protein-rich and cellulosic products are previously described.

In a first preferred embodiment of the present invention the dry rice bran is milled to less than 0.5 mm wherein an effective amount of water is added so that the water to bran ratio is about 10:1. The resulting mixture is heated to a temperature of about 50° C. and the pH of mixture is adjusted to a value of about 10.5 with use of a suitable base, preferably sodium hydroxide. The resulting solution is kept at this pH and temperature for about one hour wherein the pH is lowered to about 9 with use of a suitable acid, preferably hydrochloric acid. An effective amount of an alkaline protease, preferably alcalase, at a dosage of 10 mls/kg of protein is then added. The desired pH is maintained until a degree of hydrolysis of about 5 is reached, as measured by base addition.

In a second preferred embodiment of the present invention, the dry rice bran, is milled to an average particle size of less than 0.5 mm wherein water is added until the water to bran ratio is about 10:1. The resulting mixture is then heated to a temperature of about 60° C. and the pH adjusted to about 9 with use of a suitable base material, preferably sodium hydroxide. An effective amount of an alkaline protease, such as alcalase, is then added at a dosage of 10 mls/kg of protein. The pH of 9 is maintained until a degree of hydrolysis of 12 is reached as measured by base addition.

In a third preferred embodiment of the present invention, the instant invention is performed by milling the dry rice bran to less than 0.5 mm then adding water so that the water to grain ratio is 10:1. The resulting mixture is then heated to a temperature of about 50° C. and the pH adjusted to a value of about 11 using a suitable base material, preferably sodium hydroxide. The resulting solution is maintained at this pH and temperature for about 1 hr, then the pH is lowered to about 9 with use of a suitable acid, preferably hydrochloric acid.

In a fourth preferred embodiment and after doing any of treatments of the above first through third preferred embodiments, the pH is lowered to about 5 with use of a suitable acid material, preferably hydrochloric acid. The temperature is then adjusted to about 55° C. and an effective amount of a starch hydrolyzing enzyme, such as glucoamylase, is added to account for about 0.3% of total solids present. The pH and temperature is maintained for about 1 hr to hydrolyze the starch to glucose. The pH is then adjusted to a value of about 7.

In a fifth preferred embodiment the procedure of the above first through third embodiments is followed, but after the milling and water addition steps, the pH is adjusted to about 5 and an effective amount of glucoamylase is added as described in the above fourth preferred embodiment. After the 1 hr reaction period, the pH is adjusted to a value as described in the first through third preferred embodiment and the process continues as described in those embodiments.

In a sixth preferred embodiment the milling step in the first through third preferred embodiments is replaced with a hydrocavitation. Hydrocavitation is the process by which a fluid is passed through a small orifice to create controlled cavitation of the fluid resulting in localized high pressure and temperature. This process can disrupt and rupture cell bodies, opening up the cell structure and making it easier to solubilize the protein, or ultrasonic, treatment step wherein the rice bran is subjected to the ultrasound waves for 120 seconds (range of 30 to 120 seconds) at a power density of 1 W/mL (range of 0.3 to 2.56 W/mL).

In a more preferred embodiment of the present invention the dry rice bran, is milled to less than 0.5 mm (500 microns) and an effective amount of water is added so that the water to bran ratio is about 9:1 to about 10:1. The resulting mixture is heated to about 70° C. and the pH of the resulting mixture is adjusted to a value of about 7.5 with use of a suitable acid or basic material, depending on the natural pH of the starting material. An effective amount of a starch hydrolyzing enzyme, preferably glucoamylase, is added at 0.3% of total solids present and the temperature and pH maintained for about 1 hr to hydrolyze the starch to glucose. The pH is adjusted to 9 and the temperature raised to about 60° C. after 1 hr. An effective amount of a suitable alkaline protease, preferably alcalase, at a dosage of 10 mls/kg of protein is added. The pH and temperature are maintained until a degree of hydrolysis of about 3-4.5 is reached as measured by base addition. After the degree of hydrolysis is reached, the separation steps begin. Preferably glucoamylase is used to hydrolyze the starch to glucose. Fungal alpha amylase can be used to produce disaccharides and bacterial alpha amylase can be used for liquefaction of gelatinized starch prior to alpha amylase or glucoamylase treatment.

In another more preferred embodiment, dry rice bran is mixed with water so that the water to bran ratio is about 10:1. The mixture is then heated to 70° C. and held at that temperature. The pH of the mixture is brought to a value of 7.5 with an appropriate base or acid such as sodium hydroxide or hydrochloric acid. A starch hydrolyzing amylase enzyme, such as glucoamylase, is added at 0.3% of total solids present. The temperature and pH is kept substantially constant for about 1 hr to hydrolyze the starch to glucose. After 1 hr, the temperature of the mixture is brought to about 60° C. and a pH to 9 using sodium hydroxide. An acceptable alkaline protease is added, such as alcalase, at a dosage of 10 mls/kg of protein. Keep at the desired pH until a degree of hydrolysis of 3-4.5 is reached as measured by base addition.

What is claimed is:

1. A process for producing a protein product suitable for human consumption and a cellulosic product suitable as a feedstock for thermochemical processing from rice bran containing a starch component and a protein component, which process comprises:
a) introducing into a hydrolysis reactor, with constant stirring, rice bran an effective amount of water, so that a slurry is formed having a ratio of water to rice bran, by weight, of from about 8:1 to about 11:1;
b) heating said slurry to a temperature from about 30° C. to about 70° C.;
c) providing that the pH of the slurry be in the range from about 7 to about 10.5;
d) introducing into said hydrolysis reactor an effective amount of alkaline protease enzyme;
e) maintaining hydrolyzing condition, including temperatures from about 30° C. to about 70° C. and a pH of about 7 to 10.5, for an effective amount of time to allow the degree of hydrolysis of proteins to reach between 1 to 10;
f) raising the temperature an effective amount to result in deactivating the protease enzyme;
g) conducting the slurry resulting from step f) above to a liquid/solids separation stage resulting in a wet-solids fraction comprised of cellulosic residue material, and a liquid fraction comprised of water, protein, oils, sugars, fiber and ash;
h) conducting said aqueous fraction to a membrane microfiltration stage containing a membrane capable of performing a separation in the 20 to 500 kDa size range thereby resulting in a retentate comprised of cellulosic residue material and a permeate comprised of water and hydrolyzed protein products;
i) conducting said permeate to a membrane nanofiltration stage containing a membrane capable of performing a separation in the 500 to 1500 Daltons size range, thereby resulting in a retentate comprised of hydrolyzed protein products, and a permeate comprised of an aqueous solution of water-soluble constituents, including monosaccharides, resulting from the above process steps;
j) spray drying said retentate resulting in a spray dried hydrolyzed protein product; and
k) drying said wet-solids fraction resulting from step g) above to produce a dried cellulosic residue product.

2. The process of claim 1 wherein the rice bran is defatted rice bran.

3. The process of claim 1 wherein the average particle size of the rice bran is less than 500 microns.

4. The process of claim 1 wherein the effective amount of water is about 9 to 1 to 10 to 1 water to rice bran by weight.

5. The process of claim 1 wherein the pH of the slurry in step b) is from about 6.5 to about 7.5.

6. The process of claim 1 wherein between steps c) and d) at least 90 wt. % to 99 wt. % of the starch of the rice bran is hydrolyzed to monosaccharides by adjusting the pH of the slurry to 4.5 to 8 and an effective amount of starch hydrolyzing enzyme.

7. The process of claim 5 wherein the starch hydrolyzing enzyme is an amylase enzyme.

8. The process of claim 6 wherein the amylase enzyme is selected from the group consisting of fungal alpha-amylase, bacterial alpha-amylase, and fungal glucoamylase.

9. The process of claim 1 wherein the alkaline protease enzyme is alcalase.

10. The process of claim 1 wherein the water/rice bran slurry of step a) is subjected to ultrasonic energy prior to being treated with a hydrolyzing enzyme.
